(19) United States
(12) Patent Application Publication
(10) Pub. No.: US 2006/0223775 A1
(43) Pub. Date: Oct. 5, 2006

(54) Nucleotide sequence for treating cancer and infection

(76) Inventor: Marc Zeicher, Brussels (BE)

Correspondence Address: KNOBBE MARTENS OLSON & BEAR LLP, 2040 MAIN STREET, FOURTEENTH FLOOR, IRVINE, CA, 92614, US

(21) Appl. No.: 11/386,531

(22) Filed: Mar. 21, 2006

Related U.S. Application Data
(63) Continuation of application No. 08/807,500, filed on Feb. 27, 1997, now abandoned.
Application No. 08/807,500 is a continuation-in-part of application No. 08/448,590, filed on Sep. 28, 1995, now abandoned.

(30) Foreign Application Priority Data
Dec. 10, 1992 (BE) 09201087

(51) Int. Cl. A61K 48/00; C07H 21/02

(52) U.S. Cl. 514/44; 536/23.72

(57) ABSTRACT

A nucleotide sequence comprising the nucleotide sequence of a virus belonging to the group of autonomous parvoviruses, and at least one effector nucleotide sequence which encodes an effector polypeptide capable of effecting the destruction or the normalization of cancer cells or cells infected by virus, bacteria, or intra-cellular infectious parasites.

### BACKGROUND OF THE INVENTION

This application for patent is a continuation of Ser. No. 08/807,500, filed Feb. 27, 1997, which is a continuation-in-part of Ser. No.08/448,590, filed Sep. 28, 1995. This application is related to PCT application PCT/BE93/00080, filed Dec. 10, 1993, and Belgium application 09201087, filed Dec. 10, 1992. The entire contents of all of these applications are incorporated herein by reference.

The present invention relates to a nucleotide sequence for treating cancerous or infected cells.

The present invention also relates to the vector comprising the sequence according to the present invention, as well as a pharmaceutical composition comprising the said sequence and/or the said vector.

The invention also extends to the use of the nucleotide sequence and/or of the vector according to the invention for the preparation of a medicinal product for treating cancerous or infected cells.

The efficacy of conventional treatments of cancer and infection is limited by their lack of selectivity. Indeed, the toxicity linked to these treatments is not limited to the target cells (tumor cells, infected cells); it also affects the normal cells of vital importance.

Consequently, systems for targeting therapeutic agents have been developed which make it possible to reduce the toxic dose administered to normal tissues while allowing an effective toxic dose to be administered to the pathological tissues.

U.S. Pat. No. 4,675,382 describes hybrid proteins which are cleavable and which consist of cytotoxic fragments and cytospecific ligands, as well as their targeted therapeutic application in the treatment of medical disorders.

Proteins which are highly cytotoxic when they are introduced into mammalian cells are produced by many species of bacteria and plants (fragment A of diphtheria toxin (DT-A), from Pseudomonas aeruginosa, Ricin . . . ).

Attempts have been made to replace the portion of these proteins responsible for cell entry with tumor-specific ligands (monoclonal antibodies, peptides . . . ) (Martinez et al, Cancer Surveys, 1, 374 (1982)).

Another strategy used by Maxwell (Cancer Research, 46, 4660-4664, September 1986), consists in introducing the DNA encoding fragment A of diphtheria toxin in vitro into cells with the aid of constructs containing tissue-specific transcriptional regulatory elements which act in cis with the aim of expressing DTA only in the cells containing the factors which act on these regulatory elements.

Other techniques comprise genes encoding an enzyme which confers on the transfected cell sensitivity to a toxic agent have been described by Moolten F. (Human Gene Therapy 1: 125-134 (1990) and Venkatesh (P.N.A.S., Vol. 87, p. 8746-8750, November 1990).

The gene encoding Herpes simplex type 1 Thymidine kinase (HSV-TK) is appropriate for this type of therapy. Some guanosine analogs such as iodovinyldeoxyuridine (IVDU), acyclovir, ganciclovir are substrates specific for HSV-TK, which catalyzes their phosphorylation to monophosphate more efficiently (a thousand times more) than the thymidine kinases of mammalian cells. A subsequent phosphorylation to triphosphates under the influence of cellular kinases converts these molecules to potent inhibitors of DNA synthesis. Ganciclovir doses which do not affect the in vitro survival of HSV-TK negative cells make it possible to destroy in vitro HSV-TK positive cells and to eradicate HSV-TK positive lymphomas in vivo in transgenic mice expressing HSV-TK in their lymphoid cells.

However, for some cell lines expressing HSV-TK, the dose of guanosine analogs permitting an in vitro cytotoxicity of close to 100% cell death induces substantial cytotoxicity in cell lines not expressing HSV-TK (doses between 10 and 100 μM).

An approach which makes it possible to avoid this problem is to use guanosine analogs labeled with the aid of radioisotopes which emit AUGER electrons such as ¹²³lodine (half-life 13 h 21 min). These isotopes release most of their energy over a distance of a few nanometers. The efficiency of such radioisotopes as regards cell cytotoxicity is completely lost when they are not bound or at the very most at a distance of a few nanometers from the DNA. If they are in the vicinity of the DNA, about fifty disintegrations are sufficient to kill the HSV-TK positive cell. A maximum cytotoxicity is obtained when the cells incorporate the ¹²³Iodine labeled guanosine analog into their DNA at doses of less than 10⁻¹⁰ M which is substantially less than the toxicity threshold of this analog for cells not possessing HSV-TK.

In addition, ¹²³Iodine also emits a γ radiation which can be detected with the aid of a gamma camera in clinical medicine.

After in vivo concentration of the labeled analog in the tissues expressing HSV-TK and the elimination of the guanosine analog from the blood stream and the other tissues, there is a possibility of detecting with the aid of a gamma camera the tissues expressing HSV-TK (application to the detection of metastases).

This approach, compared with the expression of fragment A of diphtheria toxin, has the advantage of allowing control of the intensity and the course of the cytotoxic attack.

The administration of the guanosine analog can be modulated as a function of the clinical development (development of the cancerous tissues and toxicity inflicted on the normal tissues).

Gene therapy can also use genes generating after transcription RNAs which inhibit the expression of an oncogene involved in the tumor infection.

The concept of using antisense sequences of nucleic acids to block the function of messenger RNA with which they hybridize developed during the last decade. The inhibitory action of the antisense molecules on the gene expression depends either on the stability of the antisense RNA-target RNA hybrid or on the capacity of the antisense DNA-target RNA hybrid to induce an enzymatic protein destruction of the target RNA.

Likewise, it has been demonstrated that the RNA itself could have an enzymatic activity and that the catalytic RNA molecules (ribozymes) could be modified to create antisense units which, through their specific binding to the target RNA molecules catalyze their cleavage.

Thus, Haseloff and Gerlach (Nature, 334 (1988), p. 585-591) fused the catalytic region of “hammerhead” ribozymes to 2 antisense RNAs recognizing the target sequence of an RNA to be cleaved.

More recently, Herschlag (Nature, vol. 344, p. 405, 1990) described certain ribozymes which can also specifically cleave target DNAs and that there was a means of selecting in vitro mutant ribozymes which cleave DNA more efficiently than the wild-type enzyme.

In vitro, the inhibition by antisense RNA of the E6 and E7 genes (encoding the E6 and E7 oncogenes in the cells of cervical cancers associated with an infection by human papillomaviruses HPV16, 18, 31 and 33) induces a reversion of the cancerous phenotype to the normal phenotype in cervical cancer lines (Schlegel . . . ).

In addition, it has been demonstrated that RNAs can be selected from nucleotide sequence libraries in order to obtain RNAs with specific enzymatic activity or RNAs used as specific ligands.

The use of genes encoding a protein which increases the defence mechanisms of the host has also been described.

In Patent Application WO 90/11359, Baltimore et al. describe recombinant nucleotide constructs containing regulatory sequences of the HIV virus and sequences encoding cytotoxic products which specifically affect the cells infected by the HIV or HTLV viruses.

The conserved viral sequence consists of all or part of the LTR (Long Terminal Repeat) regulatory sequence of the virus. This sequence can be transactivated at the level of a TAR promoter sequence by the specific viral transactivation factors TAT of the HIV viruses and TAX of the HTLV viruses, expressed in the cells infected by these viruses (Sodroski J. et al., Science (1985) 227, 171-173).

The activation of the promoter sequence then allows the expression of the sequence encoding cytotoxic products which bring about the death of the cells infected by the HIV virus.

However, such nucleotide constructs may contain LTR sequences other than the TAR sequence.

These sequences, such as the enhancer elements NF-Kappa B (Greene, Annual Rev. Immunol., 1990, 8, p. 453) are transactivable by cellular activators, or such as the NRE element (Negative Regulatory Element) are transactivable by the viral factor NEF (Kieny, Journal of Acquired Immune Deficiency Syndromes, 3, p. 395 (1990)).

Such factors can therefore activate the cytotoxic genes in healthy cells or inactivate them in cells infected by the virus.

Patent Application WO 90/07936 and the document “Aids Research and Human Retroviruses” (vol. 8, no. 1, 1992, Mary Ann Liebert, Inc., Publisher) describe the integration, into viral vectors or plasmids, of nucleotide constructs containing regulatory sequences transactivable by factors specific for an infection (such as AIDS) and sequences encoding cytotoxic products which specifically affect cells subjected to hyperproliferative disorders or to infections.

Patent Application WO91/18088 describes the use of adenoassociated parvoviruses with low transduction efficiency (0.5 to 1.5% for a multiplicity of infection (MoI) of 1 to 10) which have, in addition, the disadvantage of not being oncoselective and of integrating into the genome of the transduced cells.

Accordingly, the technique described in this document requires an in vitro modification of hematopoietic cells before being reinjected after transduction into the patient.

Patent Application WO90/05538 describes a method of producing empty capsids of autonomous parvoviruses for:
vaccination with the aid of empty capsids;
diagnosis to detect anti-capsid antibodies;
encapsidation of the genetic material into empty capsids and introduction ofthis material into target cells. The authors envisage here the encapsidation of heterologous genetic material (not derived from autonomous parvoviruses), the example cited being parvovirus adenoassociated vectors. The parvovirus described (B19) is a pathogenic autonomous parvovirus which is expressed selectively in hematopoietic stem cells. The empty capsid is then used to correct genetic deficiencies in hematopoietic stem cells.

The document Virology (vol.186, No. 1, p.207-218) describes a Densovirus (a parvovirus which infects only insects). This virus is not infectious for mammalian cells and therefore cannot in any case be used for the gene therapy of cancer or of infection in man.

The document (Proc. Natl. Acad. of Science, USA (1990), vol. 87, p. 8746-8750) recommends the use of adenoviral vectors for the treatment of HIV infections. These adenoviral vectors still contain a substantial part of the adenovirus genome and recently their use as vector for gene therapy of cystic fibrosis has caused serious inflammatory reactions. The regulatory and effector sequences used have furthermore the following disadvantages:
the LTR sequence used stretches from −640 to +80 and should therefore confer a very high basal activity in the absence of tat;
the fragment (1084 bp) which should allow the regulation by Rev is particularly short and according to the authors does not confer regulation by Rev.

In addition, the HSV thymidine kinase used as effector sequence confers a “bystander effect” which risks killing the neighbouring uninfected cells.

Patent Application WO-A-8,808,450 is based on the addition to human stem cells, after their isolation in vitro, of a gene with therapeutic virtues and their reinjection in vivo into the patient. Among the different methods of introducing a heterologous gene into stem cells, the authors envisage parvoviral vectors, namely adenoassociated parvoviral vectors.

Likewise, Patent Applications WO-A-9007936, WO-A-9012087 and WO-A-9102805 describe viral vectors such as retroviruses, adenoviruses or adenoassociated viruses for gene therapy.

However, these viral or plasmid vectors present a potential danger on integrating into the host cells of activating protooncogenes or of annihilating tumor suppressor genes (antioncogenes).

### SUMMARY OF THE INVENTION

An object of the invention is to provide a viral nucleotide sequence containing a nucleotide sequence capable of destroying or of normalizing cancerous or infected cells.

Another object of the invention consists in providing a viral vector which, without integrating into their genome, is capable of being efficiently expressed in the intracellular environment of the said cells.

### BRIEF DESCRIPTION OF THE FIGURES

FIG. 1 represents a schematic view of the nucleotide sequence of the invention.

FIG. 2 represents the growth curves (number of cells×10⁻⁴) for normal human fibroblasts (NHF) and for transformed fibroblasts (KMST-6) as a function of time (day after infection).

FIG. 3 represents the expression of CAT (Pg CAT/10 μg of cellular extract) in these two cell lines as a fumction of time (day after infection).

FIG. 4 represents the expression of the CAT protein after two days of infection of different normal cell lines (white rectangle) or transformed cell lines (shaded rectangle) (a=fibroblasts, b=epithelial cells, c=T lymphocytes, d=B lymphocytes, e=macrophages)

FIG. 5 represents NBK cells infected with recombinant parvovirus express in B7 molecules wherein NBK 324 cells were incubated in PBS (D) or infected by MVM-B7-1(A), MVM-B7-2(B) or MVM-B7-2 antisense(C) at a MOI of 3, and analyzed for B7 expression for 48 hours later using fluoresceinated anti-B7-1(A,D) or anit-B7-2 mAbs(B,C). The table represents the percentage of cells expressing B7 at indicatd fluorescence intensity.

FIG. 6 represents the P815 cells infected with MVM-B7-1 and/or MVM-B7-2 induce proliferation of allogeneic T cells 5×10³ irradiated P815 infected with MVM-B7-1, MVM-B7-2, or both, were cultured with 2×10⁵ T lymphocytes from CBA mice. Controls include P815 cells uninfected (control) or infected with MVM-B7-2 antisense. Proliferation was assessed by thymidine incorporation during the last 16 hours of a 3-day culture. mABS specific for anti-B7-1 and/or anti-B7-2 were added in some cultures as indicated.

FIG. 7 represents P815 cells infected with MVM-B7-1 and/or MVM-B7-2 induce IL-2 secretion by allogeneic T cells. Various numbers of irradiated P815 infected with MVM-B7-1, MVM-B7-2, or both were cultured with 2×10⁵ T lymphocytes from CBA mice. Controls include P815 cells uninfected (mock) or infected with MVM-B7-2 antisense. IL-2 production was quantified from the 24-h culture supernatants. Cells were incubated in the absence (A) or presence of blocking antibodies specific for B7-1(B), B7-2(C) or with both mAbs(D).

FIGS. 8 and 9 represent the result of efficiency and selectivity of cell killing mediated by MVM/P387 TK virion and gancyclovir upon infecting normal (NHF) and transformed (MVK) human fibroblasts.

FIG. 10 represents the constriction of the plasmid ptTA neo.

FIGS. 11 and 12 represent the constriction of the plasmid pPopVPdhfr.

### DETAILED DESCRIPTION OF THE INVENTION

The invention relates to a nucleotide sequence for treating cancerous or infected cells comprising, integrated into the nucleotide sequence of a virus belonging to the group of autonomous parvoviruses, an effector sequence capable of bringing about the destruction or the normalization of the cells.

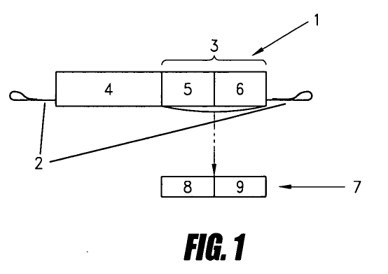

In a preferred embodiment of the invention, FIG. 1 represents a nucleotide sequence (1) according to the invention and the polypeptide sequence (7) encoded by the said nucleotide sequence (1). This nucleotide sequence (1) comprises, integrated in an autonomous parvoviral vector (2), under the control of at least one regulatory sequence (4), at least one effector sequence (3). The said effector sequence (3) preferably consists of several coding polycistronic subunits (5, 6). The said effector sequence (3) preferably encodes a fusion polypeptide (7) comprising at least one ligand (8) and an effector polypepide sequence proper (9) such as a cytotoxic polypeptide.

The term “treatment” is understood to mean a reduction or a decrease in the symptoms of the condition, the elimination or the inhibition of the causative agents, the prevention of cell infection or disorder in subjects suffering from the condition.

In the following specification, the terms “treated cell” and “target cell” refer to the cell of a patient (normal cell, cancerous and/or infected cell) which were submitted to the in-vivo, in-vitro or ex-vivo treatment of the present invention and put in contact with the nucleotide and/or vector according to the invention.

The term “infection” relates to infections of cells by viral or bacterial agents or by other intracellular infectious parasites (like the plasmodium or the trypanosoma).

These viral agents are more particularly viral infectious agents such as HIV-I, HIV-II, HIV-III, HTLV-I, HTLV-II, herpes simplex virus (HSV), cytomegalovirus (CMV), human papillomaviruses (HPV) and other similar viruses.

The viruses used according to the invention are viruses belonging to the group of autonomous parvoviruses which are preferably oncoselective. They are small viruses, with no envelope, whose genome consists of a linear single-stranded DNA of about 5 KD. Their low genetic complexity makes them totally dependent on exogenous factors for their replication. They replicate efficiently only in the intracellular environment of tumor cells in which they exist in episome form (J. Rommelaere, Handbook of Parvoviruses, 1990, vol.2, p.41-57, CRC Press Inc., Boca Raton, Fla.).

They do not become integrated into the genome of cells like retroviruses and do not therefore present the potential danger of activating protooncogenes or of annihilating tumor suppressor genes. Their oncotropism and their episomal character make them preferred vectors for targeted gene therapy of tumors and of intracellular infections.

Preferably, the viral vector used is the Parvovirus H1, the fibrotropic parvovirus of “minute virus of mice” (MVMp) or the Parvovirus Lu III.

Most human cancer cell lines tested up until now have proved to be permissive for infection by H1 parvoviruses and the fibrotropic variant of the “minute virus of mice (MVMp). These viruses have oncoprotective properties in vivo and can cause the regression of human tumors in nude mice even after viral innoculation at a site distant from the tumor. H1 and MVMp are small viruses containing a single-stranded DNA of 5 kb bordered by 2 hairpin palindromic loops.

In these parvoviruses, two promoters, P4 and P38, respectively control the expression of two nonstructural proteins (NSI and NSII) and of two capsid proteins (VP1 and VP2). P4 controls NSI and NSII and P38, VP1 and VP2. NSI is a P38 transactivation factor and is responsible for the cytotoxic activity. NSI is in addition necessary for the viral replication and the restriction or permissivity for the viral infection appears to be linked at the level of the transcription of NSI.

According to a preferred embodiment of the invention, the viral vector lacks genes encoding the capsid proteins (VP1 and VP2) of the parvovirus. Preferably, the viral vector also lacks the P38 promoter and the genes encoding the nonstructural proteins NSI and NSII of the Parvovirus.

Preferably, the autonomous parvovirus pMM 984, described by Merchlinsky et al. (J. of Virology, 47, p.227-232 (1983) is used.

The expression “bringing about the destruction or the normalization of the cells” means that during an infection or in the course of a cancer, the expression of the effector sequence in the host cell makes it possible either to kill the latter, or to make it sensitive to a toxic exogenous agent or to make it capable of inhibiting the action of the cancer or of the infectious agent.

The term “effector sequence” relates to a nucleotide sequence which, when it is expressed, for example when, under the action of specific transactivation factors acting on a regulatory sequence, it encodes an effector polypeptide capable of destroying or of normalizing the treated cells.

Likewise, this effector sequence, when it is transcribed into an RNA, for example an antisense RNA or a ribozyme, is capable of destroying or of normalizing the treated cells.

For example, a ribozyme can be obtained in order to specifically destroy the RNA transcript resulting from the fusion of exon 3 of the bcr gene with exon 2 of the abl gene, which is encountered in chronic myeloid leukemia cells Szczylik et al. (Science, 253, 562 (1991)) have been able to show that anti-bcr-abl antisense RNAs inhibited the proliferation of chronic myeloid leukemia cells.

The DNA encoding this ribozyme can be placed at the Ase I restriction site which exists in the small intron of the MVMp genome. During the splicing of the messenger RNAs of MVMp, this ribozyme will be released by the spliceosome into a nuclear compartment rich in target RNA.

The stability of such ribozymes can be increased by the addition in 3′ of a sequence such as the transcription terminator sequence of the T7 bacteriophage. This sequence will protect the ribozyme from digestion by 3′ exonucleases (Sioud et al., J. Mol. Biol. (1992), 223, p. 831-835).

The nucleotide sequence according to the invention comprising a ribozyme can be advantageously used upon leukemia because leukemic cells are easily reached via intravenous injection.

Indeed, a parvovirus containing 100 bp ribozyme sequence inserted in a small intron (site Ase I) could still replicate in malignant cells and reinfect other malignant cells after lysis of the infected cells. That replication had between 10 and 30% of the efficiency of the wild type virus. Various models (Chronic myelocytic leukemia and the bcr/abl mRNA as target) were previously described and efficient ribozymes in the nucleotide sequence according to the invention and pre-clinical models (K562 cells in SCID mice) are available.

The nucleotide sequence according to the invention is a naked DNA which is easily available (high titre) and may be accepted by ethical committees for clinical treatments upon humans.

In addition, said naked DNA could be incorporated into other vector than the natural parvoviral vector such as vaccinia vector or adenovirus vector.

The size of the parvoviral DNA can be increased by about 100 nucleotides while still preserving the encapsidation capacity and the infectivity of the parvoviruses formed. Such a ribozyme-containing recombinant virus has the ability to reproduce in tumor tissue while destroying it and can reinfect other tumor cells, develop therein and kill them.

Preferably, the effector sequence consists of several coding or noncoding nucleotide sequences arranged in polycistronic subunits, under the control of a single promoter unit.

The transfection of tumor cells by nucleotide sequences encoding for different biologically active molecules (cytokines, membrane proteins involved in immune recognition, . . . ) and the reinjection of these irradiated cells into tumor-carrying animals has been able, under certain experimental conditions, to cause the specific rejection of tumors identical to the transfected cells (Pardoll et al., Immunology Today, vol. 14, No. 6, p. 310-316 (1993)).

However, most of these molecules in addition to their property of increasing the immunogenicity of the tumors, stimulate tumor neovascularization. Accordingly, the effector sequence also comprises sequences encoding molecules which inhibit tumor neoangiogenesis (Billington D.C., Drug design and discovery, vol. 8, p. 3-35 (1991)) such as Interferon-α, Interferon-β or Platelet Factor 4 (Maione et al., Cancer Research, 51, p. 2077-2083 (1991)).

Several effector genes of the same effector sequence can be inserted into the same vector by arranging them in polycistronic units where the different genes are linked by nucleotide sequences allowing their expression following the activation of a single promoter situated upstream of the first effector gene. Among the nucleotide sequences possessing this property, there are especially the IRESs (Internal Ribosome Entry Sites) (Tzukiyama-Kohara, J. of Virology, p. 1476-1488 (1992)).

The use of autonomous parvoviral vectors which are preferably oncoselective makes it possible to cause the tumor cells of the patient's cancerous tissues to synthesize directly in vivo the different molecules promoting the rejection of the cancerous tissues. This represents an enormous progress compared with the method described by Pardoll where a piece of the patient's tumor has to be excised, made into a cellular suspension, transfected, irradiated and reinjected into the patient.

Advantageously, the nucleotide sequence according to the invention further comprises a promoter specifically activated in the target cell (possibly a normal or infected cell of a specific tissue, such as the liver, the pancreas, etc., as hereafter described) and which is inserted between the promoter P4 and non-structural protein NSI. Preferably, said promoter is the CMV promoter or the LTR promoter.

Advantageously, the effector nucleotide sequence or a portion thereof encodes at least one fusion polypeptide containing especially a (secretable) ligand, such as the hypervariable end specific of an antibody (single chain antibody), a cytokine or a growth factor, binding specifically to at least one molecule expressed at the surface of cancerous or infected cells.

In this manner, the fraction of target cells which have been infected by the recombinant autonomous parvoviruses will synthesize and will secrete the fusion polypeptides (immunotoxins, immunocytokines, immunoenzymes, . . . ) in large quantities. These fusion polypeptides will become attached mainly to uninfected cancerous cells and this process will make it possible to increase the “bystander” effect and the effect at a distance, of the gene therapy.

According to a preferred embodiment of the invention, the effector sequence or a portion of the effector sequence encodes:
at least one polypeptide or fragment(s) of this cytotoxic polypeptide, preferably fragment A of diphtheria toxin (DTA);
a molecule, such as an enzyme, conferring on a cell a sensitivity to a toxic agent, the said molecule being preferably thymidine kinase from Herpes simplex virus type 1 (HSV-TK) and the toxic agent a guanosine analog labeled with the aid of radioisotopes which emit Auger electrons, such as ¹²³Iodine;
at least one polypeptide or fragment(s) of this polypeptide, increasing the patient's immune response;
at least one polypeptide or fragment(s) of this polypeptide, inhibiting tumor neoangiogenesis.

Among the different available cytotoxic proteins (DT-A, RicinA, Gelonin, Pseudomonas aeruginosa Toxin) the choice of DT-A is preferred. Indeed, as the majority of the population has been vaccinated against the diphtheria toxin, any possible release of DT-A by the dead cells will be annihilated by the immune system. In addition, as DT-A lacks a site for binding to a cell receptor, it will not be able to enter into other cells.

The production of DT-A in mammalian cells results in the inhibition of all subsequent protein synthesis (by ADP ribozylation of the elongation factor-2) and consequently leads to cell death. As DTA acts catalytically and not stoichiometrically, a few DTA molecules are sufficient to kill a cell.

The A fragment released in the cells infected by HIV not only causes cell death, but also blocks any protein synthesis including the synthesis of viral proteins and thereby blocks reinfection of the neighboring cells.

Advantageously, the effector sequence can be modified for example by a site-directed mutagenesis so as to attenuate the cytotoxicity of the protein produced.

According to a preferred embodiment of the invention, the nucleotide sequence also comprises at least one regulatory sequence transactivable by transactivation factors specific for the condition treated or for the affected cell tissue, and capable of cisactivating the effector sequence.

The expression “bringing about the destruction or the normalization of the cells” means that during an infection or in the course of a cancer, the expression of the effector sequence in the host cell makes it possible either to kill the latter, or to make it sensitive to a toxic exogenous agent or to make it capable of inhibiting the action of the cancer or of the infectious agent.

The term “effector sequence” relates to a nucleotide sequence which, when it is expressed (by the action of specific transactivation factors on the regulatory sequence) makes it possible to destroy or to normalize the treated cells.

According to a preferred embodiment of the invention, the effector sequence encodes either a cytotoxic protein, preferably fragment A of diphtheria toxin (DTA), or an enzyme conferring on the transfected cell a sensitivity to a toxic agent; the enzyme will be prefer- ably thymidine kinase from Herpes simplex virus type 1 (HSV-TK).

Among the different available cytotoxic proteins (DT-A, RicinA, Gelonin, Pseudomonas aeruginosa Toxin) the choice of DT-A is preferred. Indeed, as the majority of the population has been vaccinated against the diphtheria toxin, any possible release of DT-A by the dead cells will be annihilated by the immune system. In addition, as DT-A lacks the site for binding to a cell receptor, it will not be able to enter into other cells.

The production of DT-A in mammalian cells results in the inhibition of all subsequent protein synthesis (by ADP ribozylation of the elongation factor-2) and consequently leads to cell death. As DTA acts catalytically and not stoichiometrically, a few DTA molecules are sufficient to kill a cell.

The A fragment released in the cells infected by HIV not only causes cell death, but also blocks any protein synthesis including the synthesis of viral proteins and thereby blocks reinfection of the neighboring cells.

Advantageously, the effector sequence can be modified for example by a site-directed mutagenesis so as to attenuate the cytotoxicity of the protein produced.

According to a preferred embodiment of the invention, the nucleotide sequence also comprises at least one regulatory sequence transactivable by transactivation factors specific for the condition treated or for the affected cell tissue, and capable of cisactivating the effector sequence.

The term “transactivable regulatory sequence” relates to a nucleotide sequence which is capable of responding to a specific transactivation factor and of activating in response the transcription of sequences situated in cis.

According to another embodiment of the invention, the regulatory sequence contains all or part of the LTR (Long Terminal Repeat) regulatory sequence of HIV or HTLV viruses comprising the TAR sequence (TAT responsive element).

Preferably, the LTR sequence lacks the kappa B enhancer sequence transactivable by cellular factors and/or the NRE sequence transactivable by the viral factor NEF.

The suppression of these sequences in the LTR sequence reduce, surprisingly, the expression of the nucleotide sequence of the invention in the cells not expressing the TAT protein without reducing the expression of the nucleotide sequence of the invention in the cells expressing the TAT protein.

Advantageously, the nucleotide sequence also contains a regulatory sequence consisting of all or part of the RRE sequence (Rev-Responsive Element) and of the adjacent CRS sequence of the HIV and HTLV viruses (Rosen, P.N.A.S., vol. 85, p. 2071-2075 (1988)) and of adjacent splicing sites.

The TAR sequences (situated in the LTR of the HIV and HTLV viruses and transactivable by the viral proteins TAT from HIV and TAX from HTLV) and the RRE sequences (situated in the ENV sequence of the HIV and HTLV viruses and responding to the proteins REV from HIV and REX from HTLV) and adjacent splicing sites are for example sequences which can be used for the treatment of infection by these retroviruses (Rosen G., Editorial Review, Aids 1990, 4, 499-509).

The high degree of conservation of these sequences makes it possible to treat the cells infected by the different HIV strains with the aid of the same nucleotide construct.

The effector sequence is also placed under the control of the Rev protein from HIV, because in the absence of Rev, any mRNA which contains the RRE sequence is blocked in the nuclear spliceosome and cannot be exported in the cytoplasm to the ribosomes.

According to another preferred embodiment of the invention, the regulatory sequence consists of at least one promoter “enhancer” sequence, preferably specific for cytomegalovirus and the effector sequence is transcribed into a ribozyme which specifically cleaves the messenger RNA encoding the cytomegalovirus a protein (Griffiths P., Biochem J., 241, p. 313-324 (1987)).

According to another preferred embodiment of the invention, the regulatory sequence consists of at least one promoter and/or of at least one enhancer transactivable in certain specific tissues, preferably chosen from the group consisting of:
the nucleotide sequence controlling the expression of the gene encoding α-fetoprotein (AFP), this region being transactivated only in hepatoma and choriocarcinoma cells and in rare hepatic cells (Sakai M., The Journal of Biological Chemistry, vol. 260, no. 8, p. 5055-5060 and Nakabayashi H., vol. 264, No. 1, p. 266-271);
the nucleotide sequence controlling the expression of human placental protein 11 (PP11). PP11 is expressed only in the placenta (syncythiotrophoblast) and it is not found in any normal adult tissue, but using immunohistochemistry, it is detected in 47% of breast cancers, in 67% of ovarian carcinomas, and in 38% of testicular and gastric cancers studied (Grundmann U., DNA and Cell Biology, vol. No. 9, No. 4, 1990, p. 243-250);
the nucleotide sequence controlling the expression of antigen CO-029 which is detected in carcinomas of the stomach, colon, rectum and pancreas but is not detected in normal tissues (Szala S., PNAS, vol. 87, p. 6833-6837 (1990));
the nucleotide sequence controlling the expression of antigen H23 (breast cancer-associated antigen) detected in humans in 90% of breast cancers (Tsarfaty I., Gene, 93, (1990) p. 313 to 318);
the nucleotide sequence controlling the prostatic expression of prostatic secretory protein PSP94; in prostate cancers at an advanced stage, after prostatectomy, the only tissue in the body synthesizing PSP94 is the prostatic tumor tissue (Linard C., Gene, 73 (1988), p. 479-487);
the nucleotide sequence controlling the expression of the protein pHGR11 associated with melanoma, ovarian cancer, adenocarcinoma of the colon and of the prostate; the only normal cells where pHGR11 is expressed are the granulosa cells of the ovary (Rapp G., DNA and cell Biology, vol. 9, no. 7, p. 479-485);
the nucleotide sequence controlling the expression of protein pHGR74, expressed in the testicles, the prostate, the seminal vesicle and the granulosa of the ovary;
as well as the sequences controlling the expression of proteins specific for the mammalian epithelium, for the uterine epithelium;
the nucleotide sequence controlling the expression of tyrosinase, expressed in the melanocytes and malignant melanoma (Kwon B., PNAS, vol. 84, p. 7473-7477);
the sequences controlling the expression of elastase, expressed only in the exocrine pancreas (Cell, vol. 9, 435-443 (1987));
the nucleotide sequence controlling the hypophysial expression of prolactin (Peers B., Molecular and Cellular Biology, September 1990, p. 4690-4700).

The present invention also relates to a recombinant (viral or plasmid) vector comprising the sequence or a portion of the sequence according to the invention.

The invention also relates to a pharmaceutical composition for treating cancerous or infected cells comprising at least one sequence and/or the vector according to the invention and a pharmaceutically acceptable vehicle.

According to a preferred embodiment of the invention, this pharmaceutical composition also comprises one or more wild-type viral agents belonging to the group of autonomous parvoviruses.

Another aspect of the invention relates to the use of the pharmaceutical composition, of the sequence and/or of the vector according to the invention for the preparation of a medicinal product for treating cancers and/or infections.

The examples below are given purely as a guide and make it possible to illustrate other characteristics and advantages of the present invention.

### EXAMPLE 1

### Treatment of H.I.V. Infection

The viral sequences used are, on the one hand, the 5′LTR sequence of HIV modified so as to be controlled by the HIV TAT protein and so as to escape control by cellular transactivation factors, and on the other hand, the RRE sequence placed under the control of the HIV Rev protein and linked to the adjacent CRS sequence. The effector sequence placed between these two sequences consists of the gene encoding fragment A of diphtheria toxin. These two sequences impose a double safety on the expression of the A fragment which can only occur in the cells infected by HIV which produce (even at low noise such as in latent infections) both the TAT and REV proteins. The TAR sequence (controlled by TAT) and the RRE sequence (REV responsive element) from HIV-1 respond to the TAT and REV proteins from HIV-1 and HIV-2 as well as to the TAX and REX proteins from HTL-V-1. The high degree of conservation of these sequences makes it possible to treat cells infected by the different strains of HIV with the aid of the same construct. The advantage of the parvovirus over other transfection vectors is that there is no integration into the cell genome of its genetic material, which remains in episomial form and does not risk inactivating an oncosuppressor gene or activating a protooncogene.

The safety and efficacy of such a treatment should also allow its administration to seropositive patients at the asymptomatic latent infection phase. The A fragment released in the cells infected by HIV not only causes cell death but also blocks any protein synthesis including the synthesis of viral proteins and thereby blocks reinfection of neighboring cells.

Process for Producing the Vector Construct:

1. Isolation of the Transactivable Regulatory Sequence

Amplification by PCR of the LTR fragment (−85, +78) from HIV-I and insertion into the SmaI (715 Blunt) and HindIII (689) sites (compatible with +78=HindIII site) of the plasmid p Bluescript SK +/−®.
The LTR sequence from HIV thus lacks NFKB sites but conserves 3 SP1 sites, the TATA Box and TAR (TAT Responsive Element).
   2. Isolation and Integration of the Cisactivable Effector Sequence
Amplification by PCR of the DTA fragment placed between the nucleotides (79) and (653)
formation of oligomers HindIII-ATG-(79)-DTA and DTA-(653)-TAG-KPnI
Insertion of the HindIII ATG-(79)-DTA-(653) TAG-KPnI into the HindIII (689) and KPnI (657) sites of the plasmid pBluescript SK +/− HIV LTR.
Isolation of the fragment (2668 bp): KPnI (6346)-CRS-RRE-KpnI (9014) from HIV-I.
Insertion into the KPnI site (657) of the plasmid pBluescript SK +/−LTR-DTA (insertion in both orientations).
Isolation of the fragment BamHI (Bluescript 719)-BamHI (HIV 8474) having the correct orientation (2865 bp) and another wrongly orientated fragment (1277 bp).
   3. Integration of the Effector Sequence and of the Regulatory Sequence into the Parvoviral Vector
Digestion of the vector pMM 984 (MVM vector) with NCOI (259) and XbaI (4335) add BgIII polylinker insert fragment BamHI-LTR-DTA-CRS-RRE-BamHI and select the clones having the correct orientation.
   4. Cloning of the Vector into E. coli
The clones having the correct orientation are selected and the plasmids isolated. (pMM 984 tends, when it is propagated in E. Coli, to lose 40 or 97 base pairs in the right palindrome, which is not the case in Epicurian Coli sure (Stratagene®).
   5. Production of Virions
To encapsidate the recombinant parvoviral genome, a help plasmid (either pMM 984 or a pMM 984 having a deletion in the right palindrome) is cotransfected with the recombinant plasmid selected in tumor cells.
After 2 days of culture, the supernatants (2 days per transfection) are harvested. The cells are frozen and thawed and the virions harvested and isolated on a cesium chloride gradient.
Likewise, it is also possible to use in the abovementioned process “packaging” cells constitutively or inducibly producing VP1 and VP2.

### EXAMPLE 2

### Treatment of Breast Cancer

Process for the Production of the Vector Construct

1. Isolation of the Transactivable Regulatory Sequence

Amplification by PCR of the fragment
GAG Hind III-H23 Ag (280)-H23 Ag (784)-EcoRI-GAG of 584 base pairs of the 5′ flanking sequence of H23 Ag (ATG in 785) from the genomic DNA of the human mammary cancer line (T47D).
Digestion with HindIII and EcoRI and insertion into the HindIII (689) and EcoRI (701) sites of the plasmid pIBluescript SK +/−.
   2. Isolation and Integration of the Cisactivable Effector Sequence
Amplification by PCR of the fragment
GAG EcoRI-ATG Diphtheria Toxin DTA (79-653) TAG-XbaI-GAC fragment encoding the toxic portion ofthe diphtheria toxin lacking the “hydrophobic leader signal sequence” by the primer GAG EcoRI ATG-DTA (79-100) and by the primer DTA (631-653)-TAG-XbaI-GAG
Digestion with EcoRI and XbaI and insertion of the DTA fragment into the EcoRI (701) and XbaI (731) sites of the plasmid pBluescript SK +/− containing the H23 Ag fragment.
   3. Integration of the Effector Sequence and of its Regulatory Sequence into the Parvoviral Vector:
The HindIII site of the plasmid PBR 322 into pMM 984 is destroyed by removing the ClaI-NheI fragment from PBR 322 and by making “blunt” the protruding 5′ ends in the presence of the Klenow fragment of DNA polymerase from E. Coli. The pMM 984 (HindIII PBR 322-) is then recircularized.
Digestion with HindIII and XbaI and insertion of the fragment H23-ATG-DTA TAG into the HindIII (2650) and XbaI (4339) sites of the plasmid (lacking the HindIII site of PBR 322) pMM 984 by replacing the sequences encoding the VP1 and VP2 proteins of MVMp.
The following sequence is therefore obtained from the modified MVMp:
Palindrome, promoter P4, NSI, NSII, promoter P38, promoter enhancer region H23-ATG-DTA-TAG-MVM polydenylation [sic] sites, Palindrome.
   4. Cotransfection with Wild-type Parvovirus Plasmid DNA (or Virus with Nonfunctional 5′ Palindrome) in Order to Provide the Viral Capsids (VP1 and VP2) in the Virion-producing Cells.

### EXAMPLE 3

### Vector Construct to be Used for the Treatment and Diagnosis of Cancer (Breast Cancer)

1. Isolation of the Transactivable Regulatory Sequence

The amplification by PCR of the fragment GAG HindIII-H23 Ag (280)-H23 Ag (784)-EcoRI-GAG and its insertion at the HindIII (689) and EcoRI (701) sites of the plasmid pBluescript SK +/− is performed as above.
   2. Isolation and Integration of the Cisactivable Effector Sequence
Amplification by PCR of the fragment
GAG EcoRI-ATG-HSV-1 Thymidine kinase (59 to 1189) TGA XbaI-GAG
Insertion at the EcoRI (701) and XbaI (731) sites of the plasmid pBluescript SK +/− containing the H23 Ag fragment.
   3. Integration of the Effector Sequence and of its Regulatory Sequence into the Parvoviral Vector
Insertion of the fragment H23 -Tk into the HindIII (2650) and XbaI (4339) sites of the plasmid pMM 984.
Synthesis of iodovinyldeoxyuridine labeled with ¹²³Iodine for the treatment and detection, using a gamma camera, of cancerous cells expressing HSV-I Tk after infection with the vector described above (Samuel J. et al., Int. J. Appl. Radiat. Isot., vol. 35., No. 11, p. 1049-1052 (1984)).

### EXAMPLE 4

### Vector Construct to be Used for the Treatment of Hepatoma or Choriocarcinoma

1. Isolation of the Transactivable Regulatory Sequence

Amplification by PCR of the fragment
GAG HindIII-AFP enhancer (−736, +44)-EcoRI-GAG with the primer GAG HindIII-AFP enhancer (−736, −716) and the primer AFP enhancer (+24, +44) EcoRI GAG and insertion into the HindIII (689) and EcoRI (701) sites of the plasmid pBluescript SK +/−.
   2. Isolation and Integration of the Cisactivable Effector Sequence Amplification by PCR of the fragment GAG EcoRI-ATG-DTA (79-653) TAG-XbaI -GAG and insertion into the EcoRI (701) and XbaI (731) sites of the plasmid pBluescript SK +/− containing the AFP enhancer.
   3. Insertion of the Effector Sequence and of its Regulatory Sequence into the Parvoviral Vector
Insertion of the fragment AFP enhancer ATG DTA TAG into the HindIII (2650) and XbaI (4339) sites of the plasmid pMM 984

### EXAMPLE 5

### Treatment of Cytomegalovirus Infection Occurring in Immunosuppressed Individuals

In subjects with normal immunity, viral infection is combated by cytotoxic T lymphocytes which recognize peptides derived from viral proteins which have been degraded after endogenous synthesis by the infected cells. These peptides are recognized in association with the MHC class I (Major Histocompatibility Complex) molecules. The destruction of the infected cells prevents the propagation of the viruses to the other cells by inhibiting their replication. The body's defences also comprise the intracellular production of interferon and the production of specific antibodies. In the absence of specific antiviral drugs and taking into account the limited efficacy of passive immunotherapy, it is proposed in the invention to treat immunosuppressed individuals suffering from viral conditions with the aid of parvoviruses modified by replacing the sequences encoding NS-1, NS-2, P38, VP-1 and VP-2 with a promoter “enhancer” sequence controlled by transactivation factors specific for the infecting virus. This sequence controls the expression of an effector sequence allowing the transfected cells either to resist the infection or to be eliminated.

Although cytomegalovirus infection is asymptomatic in individuals having a normal immune system, it becomes a major cause of death and morbidity in patients with either an immature (fetus, newborn) or compromised (recipients of allografts, patients suffering from AIDS) immunity. Intrauterine CMV infections are the second cause of mental retardation after Down's Syndrome (Griffiths and al., Biochem. J. (1987), 241, p. 313-324).

CMV pneumonia is the principal cause of death after bone marrow transplant and discriminate CMV infection is the major cause of morbidity and mortality in renal transplant patients or in patients suffering from AIDS.

After infection of the susceptible cell by CMV, temporal expression of the virus genome is closely controlled under the form of a cascade synthesis of messenger RNA and of proteins. The α (or immediate early), β (or retarded early and γ (or late) genes can be distinguished. The products of the α genes are required by the virus in order to take control of the syntheses of the host cell, the β products control the production of virions whereas the γ products form the structural components of the virion.

The α proteins allow the synthesis of β messenger RNA and the β proteins allow the replication of DNA which is followed by the synthesis of γ messenger RNA.

The α and β genes are transcribed by cellular RNA polymerase II. Their expression is controlled by sequences proximal in relation to the promoter which are activated in trans by a structural protein of the virion.

According to the invention, these sequences are inserted after the P4 promoter of the parvovirus and are followed by a sequence encoding a ribozymial RNA which specifically cleaves the messenger RNA encoding the most abundant a protein (Spaete and Mocarski, 1985, J. virol., 56, 135-143; Sternberg et al, 1984, J. virol., 49, 190-199). The cells transfected by the parvovirus modified in this manner are protected from infection by CMV. Indeed, during infection, the removal of the viral envelope gives rise to the structural protein which will transactivate the sequence included in the parvovirus and will initiate the production of ribozymes to mRNA of the a protein.

### EXAMPLE 6

### Vector Construct Comprising Polycistronic Units

Autonomous parvoviral vector containing the GMCSF (Granulocyte Macrophage Colony Stimulating Factor) and the PF4 (platelet factor 4) gene in the form of a bicistronic unit linked by the IRES (Internal Ribosome Entry Site) of the hepatitis C virus.

In various experimental murine systems, reinjection of irradiated tumor cells transfected with a gene encoding a cytokine (especially encoding GMCSF) allows the eradication of preexisting tumors (Pardoll, Immunology Today, p. 310-316, vol. 14, No.6(1993). However, some of these cytokines (especially GMCSF) are potent activators of tumor neovascularization. This property is probably of little importance when the irradiated cells are injected far from the tumor sites. But during the use of vectors capable of causing cytokine to be specifically expressed by the tumor tissue, this effect on neovascularization can be damaging. Accordingly, the autonomous parvoviral vector according to the invention contains both sequences encoding GMCSF and PF 4 (which is a potent inhibitor of tumor neovascularization).

Using PCR, the gene encoding GMCSF is isolated by adding to it in 5′ a HindIII site and in 3′ an NCOI site (plasmid GMCSF® from the British Technology Group), pGEM 7® (Promega) is modified by introducing into the Smal site, an NCOI site and into the EcoRI site, a BglII site. The HindIII-GMCSF-NcoI fragment is inserted into the modified plasmid pGEM 7.

A fragment including the sequence between nucleotides 101 and 332 is isolated from the untranslated 5′ region of the hepatitis C virus by PCR (Tsukiyama-Kohara, Journal of Virology, 1992, 1476-1483). This fragment comprises an NCOI site in 5′ and a BglII site in 3′ including IRES (101-332). The insertion is made into the NCOI and BglII sites of the modified pGEM 7 containing GMCSF. The fragment containing the gene encoding human PF4 is isolated by PCR (Barone, J. Biol. Chem., 263, 8710-8715 (1988)).

This fragment comprises a BglII site in 5′ and an XbaI site in 3′ including the PF4 gene.

This fragment is inserted between the BglII and XbaI sites of the modified pGEM 7 containing GMCSF and IRES.

The fragment HindIII-GMCSF-NCOI-IRES-BglII-PF4-XbaI is isolated from pGEM 7 and inserted into pMM 984 (deleted of the HindIII site (9169) between the HindIII (2650) and XbaI (4342) sites. A plasmid is thus constructed containing the GMCSF and PF4 genes which is placed under the control of the P38 promoter of the parvovirus MVMp.

### EXAMPLE 7

### Construction of an Autonomous Parvoviral Vector Containing the B7 Murine Gene

In different experimental murine systems, the reinjection of irradiated tumor cells transfected with the murine gene encoding the B7 protein allows the eradication of preexisting tumors. The B7 murine gene is inserted into the parvovirus MVMp in place of the genes encoding the capsid proteins VP1 and VP2.

The plasmid containing the B7 murine gene is obtained from the A.Z. Jette center (Vrije Universiteit te Brussel).

This gene exists therein in the form of a HindIII-XhOI fragment of 944 base pairs.

The B7 fragment (HindIII-XhOI) is introduced into the HindIII and XhOI sites of the plasmid pGEM 7® (Promega), which contains an XbaI site adjacent to the XhOI site.

Moreover, the plasmid pMM 984 containing MVMp is deleted of the HindIII site (9169) present in the pBR322 portion of pMM 984. (After partial digestion, the site 9169 is made blunt by the Klenow fragment of DNA polymerase).

The B7 gene included in a HindIII-XbaI fragment is isolated from the modified pGEM and inserted at the HindIII (2650) and XbaI (4342) sites of MVMp in the plasmid pMM 984 deleted of the HindIII site (9169).

To produce recombinant virions containing the B7 gene, the plasmid pM984 containing the B7 gene is cotransfected with a plasmid pMM984 containing an MVMp deleted of the right palindrome in COS cells. Three days after the transfection, the cells are frozen and thawed three times and the virions are harvested and isolated on a cesium chloride gradient.

The isolated virions are resuspended after dialysis in PBS pH 7.2 and used to infect different cell lines.

Normal human fibroblasts (NHF) obtained from primary culture and a transformed human fibroblast line are infected at a multiplicity of infection (M.O.I.) of about 10⁻².

The expression of the B7 protein at the surface of the infected cells is measured by immunofluorescence with the aid of a Fluorescent Activated Cell Sorter (Becton Dickinson).

The results obtained on 10,000 cells measured are the following as shown in Table A:

TABLE A

| | |
|---|---|
| | Number of cells expressing a |
| | given level of B7-specific |
| Fluorescence | fluorescence (mean |
| intensity | intensity in FACS units) |

| (FACS units) | NBK | NHF |
|---|---|---|
| >1000 | 12 (1150) | 0 |
| from 750 to 1000 | 3 (846) | 0 |
| from 500 to 750 | 11 (626) | 0 |
| from 250 to 500 | 13 (372) | 0 |
| from 100 to 250 | 28 (170) | 0 |
| from 10 to 100 | 0 | 0 |
| <10 | The remainder of the | All the cells |
| | 10000 cells | |

Table A shows that in the NBK cells, 67 cells possess a mean intensity, in FACS units, greater than 100; the mean is 490 and the median 340.

In the NHF cells, all the cells possess a mean intensity, in FACS units, of less than 10.

### EXAMPLE 8

### Construction of an Autonomous Parvoviral Vector Containing an Anti-bcr-abl ribozyme Inserted at the AseI Site (2350) of MVMp

The Asei site (8316) present in the pBR322 portion of pMM 984 is deleted. After partial digestion, the AseI 8316 site is made blunt by the Klenow fragment of DNA polymerase I.
Double-stranded DNA corresponding to an anti-bcr-abl ribozyme is synthesized (Applied Biosystems DNA Synthesizer). This double-stranded synthetic DNA has, on either side, protruding ends corresponding to the AseI restriction site.

The anti-bcr-abl ribozyme consists of the sequence described on p. 601 in the article by Snyder et al.: Blood, vol. 82, No. 2, 1993, p. 600 to 605, surrounded by 2 AseI sites.

The double-stranded DNA containing the sequence encoding the anti-bcr-abl ribozyme between two AseI sites is inserted at the AseI site (2350) of pMM 984.

After transformation of the Epicurian Coli Sure® bacteria (Stratagene) by electroporation, the positive clones (isolated by hybridization with a radioactive probe specific for the ribozyme sequence) is sequenced and the clones possessing a ribozyme in the correct orientation are isolated. The production of virions is carried out by transfection of COS cells or of NBK cells.

To stabilize the ribozyme in vivo, the sequence encoding the transcription termination signal from the T7 bacteriophage is added in 3′ to the sequence encoding the anti-bcr-abl ribozyme (Sioud et al., J. Mol. Biol. (1993), 223, p. 831-835). The whole is also inserted at the AseI site (2350) of pMM 984.

### EXAMPLE 9

### Construction of an Autonomous Parvoviral Vector Containing the CAT (Chloramphenicol Acetyl Transferase) Gene in Place of the Genes Encoding the Capsid Proteins

The fragment containing the CAT gene is inserted between a HindIII site in 5′ contiguous to ATG and an XbaI site in 3′ at the HindIII (2650) and XbaI (4342) site of the plasmid pMM 984 deleted of the HindIII (9169) site.

The production of virions is carried out by cotransfection of COS cells with the aid of the plasmid obtained (P38.CAT) and of pMM 984 deleted of the right palindrome of MVMp.

Three days after the transfection, the cells are frozen and thawed three times and the virions are harvested and isolated on a cesium chloride gradient. The isolated virions are resuspended after dialysis in order to infect different cell lines.

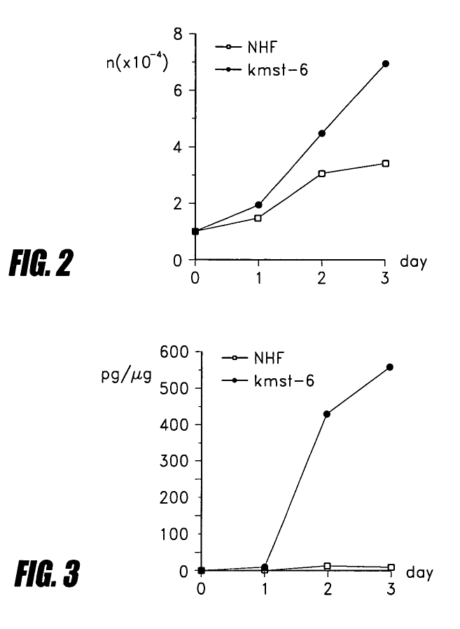

FIG. 2 shows the growth curves for normal human fibroblasts (NHF) and for human fibroblasts transformed 1, 2 and 3 days after infection with virions P38 CAT (MOI<10⁻²).

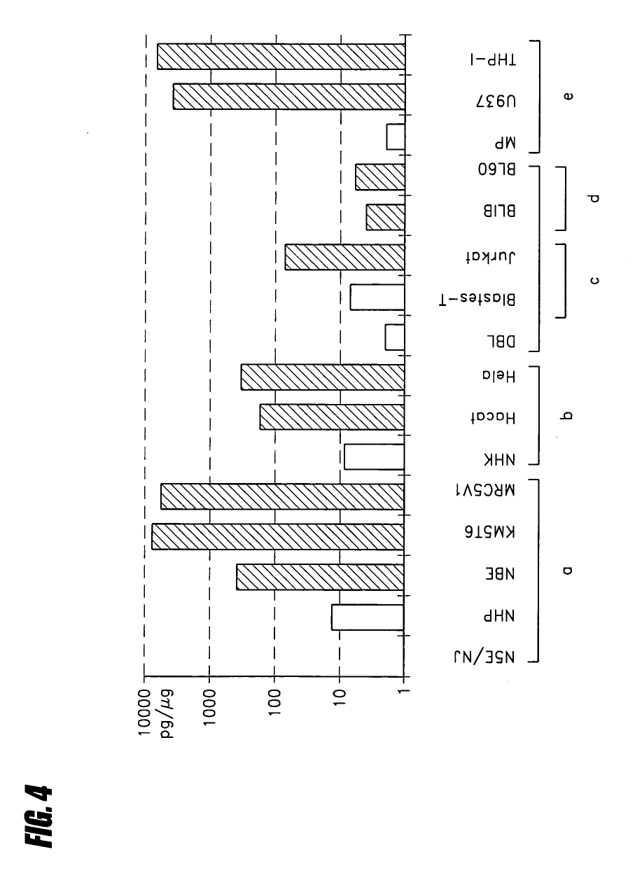

FIG. 3 shows the production of the CAT protein (measured by CAT-ELISA from Boehringer) of these same cells.

In spite of the fact that they multiply at a good rate, the normal fibroblasts only produce quantities of CAT protein at the detection limit, whereas the transformed cells produce abundant quantities thereof.

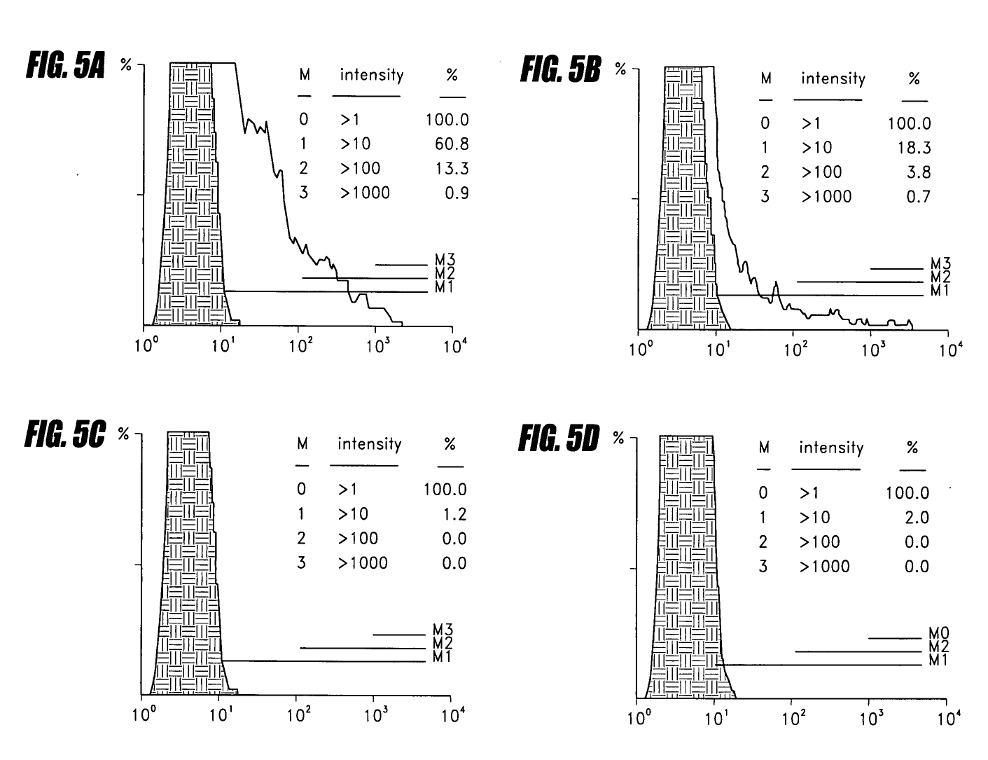

FIG. 4 shows the expression of CAT measured 2 days after infection of different normal human cell lines (in white) and of transformed cells (shaded). It can be seen that no normal cell exceeds 10 pg/10 microgram of cellular extract protein.

Apart from the B lymphomas, all the transformed cells tested express, at varying degrees, substantial quantities of CAT protein.

### EXAMPLE 10

### Results with the Vector Comprising B7 Effector Gene

The introduction of co-stimulatory molecule B7-1 and B7-2 (MVM-B7-1 and MVM-B7-2) in tumor cells in several models appears to enhance the antitumoral immune response and in all the experiments, tumor cells were transfected with the B7 genes and reinjected into tumor bearing animals.

Some conditions lead to tumor rejection, see publications of (the entire contents of all of which are incorporated herein by reference):
Li, Y. et al, Journal of Immunology 153: 421-428, (1994);
Townsend, S. E. et al, Science 259: 368-370, (1993), Cancer Research 54: 6477-6483, (1994);
Ramarathinam et al, Journal of Experimental Medicine, 179: 1205-1214, (1994).

In human clinical condition, that approach is not very practical and that is why people are looking for direct intratumoral injection of the B7 genes (naked DNA plasmids or viral vectors).

But it has been shown that the expression of B7-1 molecules in normal cells (pancreatic beta-cells) can induce auto-immune disease (diabetes) and that phenomenon is increased if viral proteins are expressed.
Wong, et al, Diabetes 44(3): 326-3298, (1995);
Guerder, et al, Immunity 1(2): 155-156, (1994),
P.N.A.S. 91(11): 5138-5142, (1994);
Von Herrath, M G et al, Immunity 3(6): 727-738, (1995);
Harlan, D. M., et al, P.N.A.S. 91: 3137-3141, (1994)

As most tumor mass are a mixture of cancerous and normal cells, the new expression of B7 molecules on normal cells can lead to auto-immune disease.

The use of a vector transducing B7 only on cancer cells without affecting normal cells would avoid that issue. The results show that the recombinant parvovirus (MVM-B7-1 and B7-2) according to the invention fulfils these requirements. Indeed, as shown in Table I, for a given multiplicity of infection (MOI) 16,9% NBK324; 7,6% MRC5V1 and 2,1% of KMST6 (NBK, MRC5V1 and KMST6 are transformed human fibroblasts) displays B7-1 antigen on their surface (obtained with FACS analyzer after subtraction of Mock-infected backgrounds) while no B7 molecule on the normal human fibroblast cell lines NHF, MRC5, KMS6 (all values below background) were detected.

TABLE 1
Expression of B7-1 molecules in transformed (bold) and
non-transformed cells infected with recombinant parvovirus.

| | FR3T3 | FREJ4 | FRMT4 | NHF | NBK | MRC-5 | MRC-5V1 | KMS6 | KMST6 |
|---|---|---|---|---|---|---|---|---|---|
| Mock-infected a | <0.1 b | <0.1 | <0.1 | 6.0 | 2.0 | 1.0 | 0.2 | 1.1 | 0.1 |
| MVM-B7-1 | <0.1 | 2.1 | 1.3 | 2.0 | 18.9 | 0.6 | 7.8 | 1.2 | 2.2 |

a Cell lines were incubated with supernatants from untransfected COS-7 (mock-infected) or COS-7 transfected with helper and B7-1 plasmids.
b Data are expressed as % cells stained with fluoresceinated anti-B7-1 mAbs.

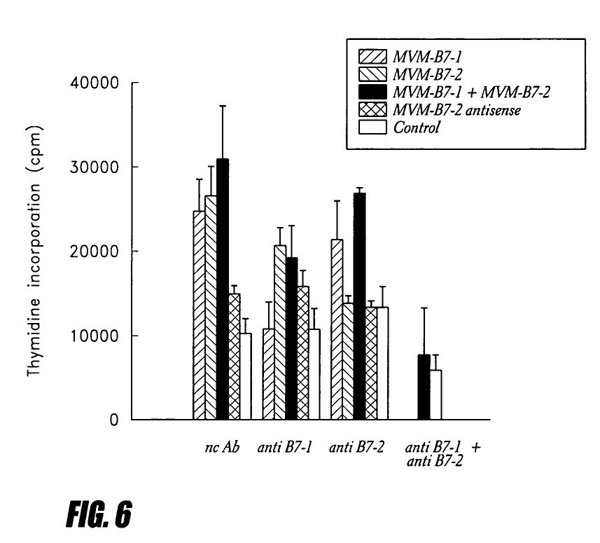

These data confirm at the single cell level the onco-selectivity of the recombinant parvovirus shown on cell lysates as measured by CAT Elisa and reveal the variability of efficacy of transduction of different cell line for the same dose of recombinant virions. Details of infections of NBK cells are shown in FIG. 5.

It confirms also at the single cell level the variability of efficacity of transduction of different cell lines for the same dose of recombinant virions. These data have been extended to cell lines derived from clinical tumor samples (biopsy) namely from breast cancer, melanoma, chronic myelocytic, leukemia, monocytic leukemia, hepatoma, firbosarcoma. Most of them are very efficiently transduced upon injection with recombinant MVM parvoviruses. The variability in efficiency is not unique to this approach. It is encountered in most anti-cancer therapies (chemotherapy, radiotherapy . . . ).

These results can be extrapolated with other parvoviral vector according to the invention such as the parvovirus HI, and the LuIII.

In the murine mastocytoma cell line P815, results show selective expression of B7-1 and B7-2 molecules upon infection with the respective MVM recombinant (see table 2).

TABLE 2
Selective expression of B7 molecules by P815 tumor
cells infected with recombinant MVM.

| Staining a/ | | | MVM-B7-2 | |
|---|---|---|---|---|
| infection b | MVM-B7-1 | MVM-B7-2 | antisense | Mock-infected |
| unstained | | | | |
| >1 c | 100 d | 100 | 100 | 100 |
| >5 | 3.4 | 4.7 | 4.9 | 3.4 |
| >10 | 0.6 | 0.8 | 0.8 | 0.6 |
| >40 | 0 | 0 | 0 | 0 |
| >100 | 0 | | 0 | 0 |
| anti-B7-1 | | | | |
| >1 | 100 | 100 | 100 | 100 |
| >5 | 26.5 | 5.3 | 4.6 | 4.3 |
| >10 | 12.4 | 0.7 | 0.6 | 0.6 |
| >40 | 2.2 | 0 | 0 | 0 |
| >100 | 0.9 | 0 | 0 | 0 |
| anti-B7-2 | | | | |
| >1 | 100 | 100 | 100 | 100 |
| >5 | 2.6 | 24.2 | 3.4 | 3.8 |
| >10 | 0.5 | 11.3 | 0.4 | 0.5 |
| >40 | 0 | 2.8 | 0 | 0 |
| >100 | 0 | 1.6 | 0 | 0 |

a. P815 cells were either unstained or stained with fluoresceinated anti-B7-1 or anti-B7-2 mAbs.
b. B815 cells were incubated with supernatants from untransfected COS-7 (mock-infected) or COS-7 transfected with helper plasmid and B7-1, B7-2 or B7-2 antisense plasmid.
c. Arbitrary gates on the logarithmic scale of green fluorescence.
d. % P815 cells expressing B7 molecule.

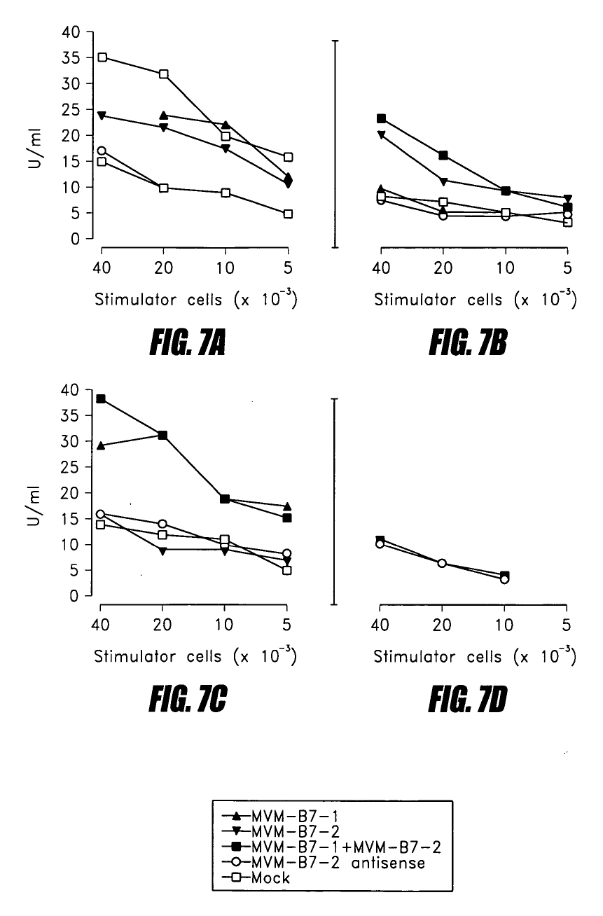

In addition, the P815 cells infected with MVM B7-1 and/or MVM B7-2 recombinant virions induce proliferation of allogeneic T-lymphocytes and that proliferation was blocked by the relevant anti-B7 antibodies (see FIG. 6).

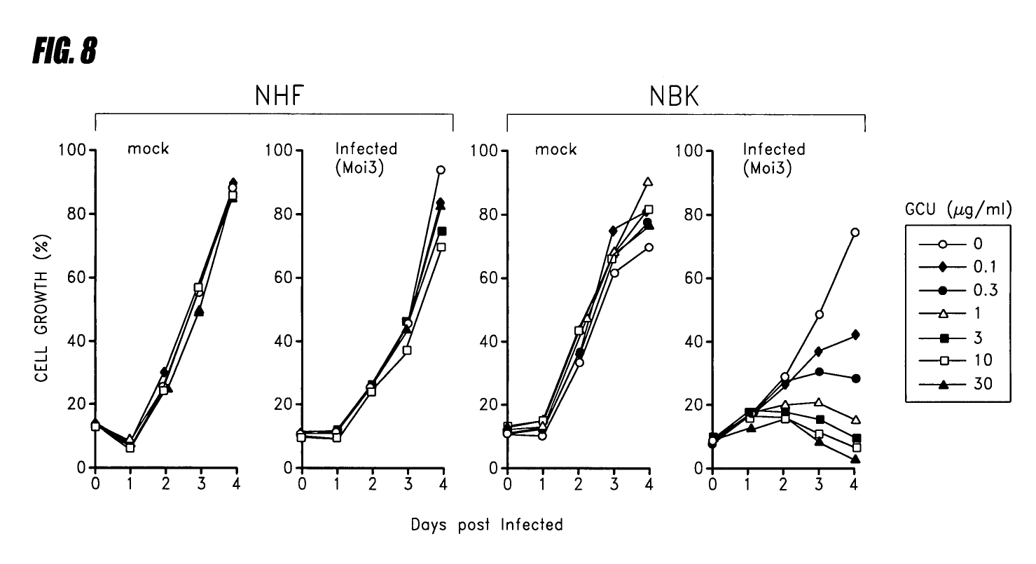

These results also show that P815 cells infected with MVM B7-1 and/or MVM B7-2 induce IL-2 secretion by allogeneic T-lymphocytes and said secretion was blocked by the relevant anti-B7 antibodies (see FIG. 7).

These experiments confirm the immunological functionality of the B7 molecule transduced into P815 cells upon infection with recombinant MVM-B7 virion.

In vivo experiments with the P815 mastocytoma in a syngeneic DBA/2 mice are currently in progress.

### EXAMPLE 11

### Results with Vector Comprising the HSV-TK Effector Gene

Another effector gene has been incorporated in an MVM recombinant vector according to the invention. The Herpes Simplex Virus Thymidine Kinase gene (HSV-TK) gene codes for a drug activating enzyme which efficiently converts the nontoxic prodrug gancyclovir (GCV), a guanosine analog into a potent inhibitor of DNA synthesis. Therefore, the HSV-TK mediated cytotoxicity is not only restricted to the transduced cell but also affects the neighbouring non infected cells via release of the phosphorylated gancyclovir through gap Junctions existing between contiguous cancer cells (bystander effect).

Thus, the majority of cells in a tumor mass could be eradicated if only a fraction of them expresses the effector gene. Retroviral vectors based upon said technology have been used in order to transduce brain tumors. In that restricted clinical situation where proliferating tumor cells are surrounded by non-dividing neural tissues, retroviral vectors are quite tumor specific (Culver, et al, Science 256: 1550-1552, 1992).

In order to achieve a real onco-selectivity that will allow to deal with clinical situations where tumor are surrounded by dividing normal cells, the inventor has developed an MVM-based vector expressing the HSV-TK gene under the control of the parvoviral P38 promoter (MVM-TK).

The 1.4 kb HSV-1-TK gene was inserted between the Hind III (2650) and XbaI (4342) sites of plasmid PMM984.

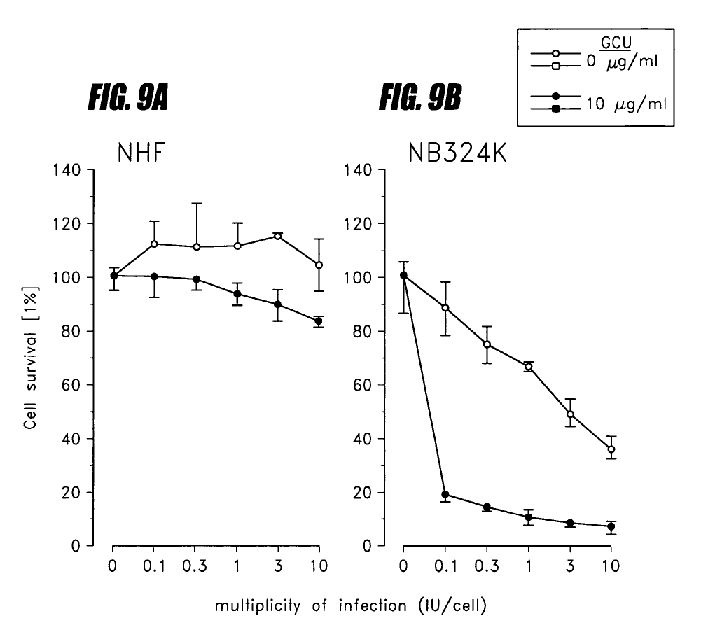

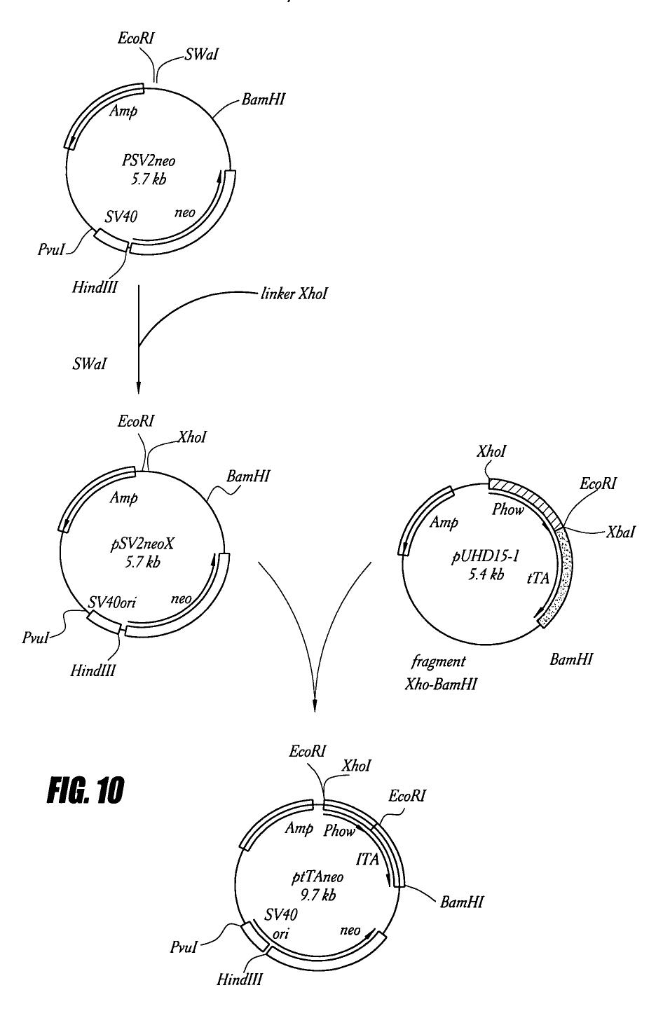

The efficiency and selectivity of cells killing mediated by the MVM/P387-TK virions and gancyclovir were assessed by infecting normal (NHF) and transformed (NBK) human fibroblast. The results of said experiments are described in the enclosed FIGS. 8 and 9.

FIG. 8 shows the effect of different concentration of GCV on mock or MVM-TK infected NHF and NBK cells. Cell growth was daily monitored with the WST-1 colorimetric assay (Boehringer). NHF and mock infected NBK were not affected by the different GCV concentrations, whereas the growth of infected NBK cell was severely suppressed by GCV concentration of 3 μg/ml or more.

In FIG. 9, NHF and NBK cells were infected at different MoI and the cell survival was assessed after 4 days with the WST-1 assay in presence or absence of GCV.

Thus, normal fibroblasts were essentially unaffected 4 days after infection at different MoI while NBK cells survival was inversely proportional to the MoI (this is due to the cytotoxic effect of the parvoviral NS protein on transformed cell). That cytotoxic effect was tremendously increased in presence of GCV even at very low MoI (0.1).

These results are the first examples of TK-transducible vector that is toxic for cancer cells without affecting normal dividing cell.

### EXAMPLE 12

### Improvement of Production of Recombinant Virions by Packaging Cell Lines

Methods of production of recombinant parvoviruses relying on co-transfection of producer cells with a helper plasmid providing in trans the capsid genes result often in a contamination with a wild type virus due to the homologuous recombinations. In order to circumvent that effect and to improve the yield of production, inducible packaging cell lines have been developed.

This method is based on the system described by Gossen and Bujard, (P.N.A.S. 89: 5547-5551, (1992)) which describe a highly efficient regulatory system in mammalian cells based on the control elements of the tetracycline-resistance operon of E.Coli. By fusing the tet repressor with the activating domain of Virion Protein 16 of the Herpex Simplex Virus, a tetracycline-controlled transactivator (tTA) was generated. This transactivator stimulates transcription from a minimal promoter sequence derived from the human cytomegalovirus promoter IE combined with the tet operator sequences. Upon transfection of a luciferase gene controlled by a tTA-dependant promoter into a tTA producing cell line, high levels of luciferase expression were monitored. That production was negatively regulated by tetracycline.

The starting point cell line used was the NBK 324 cell line known to be the best producer of wild type MVM virus.

The NBK 324 cell line was stably transfected with the plasmid ptTA neo (expression vector containing under the control of CMV promoter the gene coding for tTA and under the control of RSV promoter, the gene coding for the resistance to neomycine).

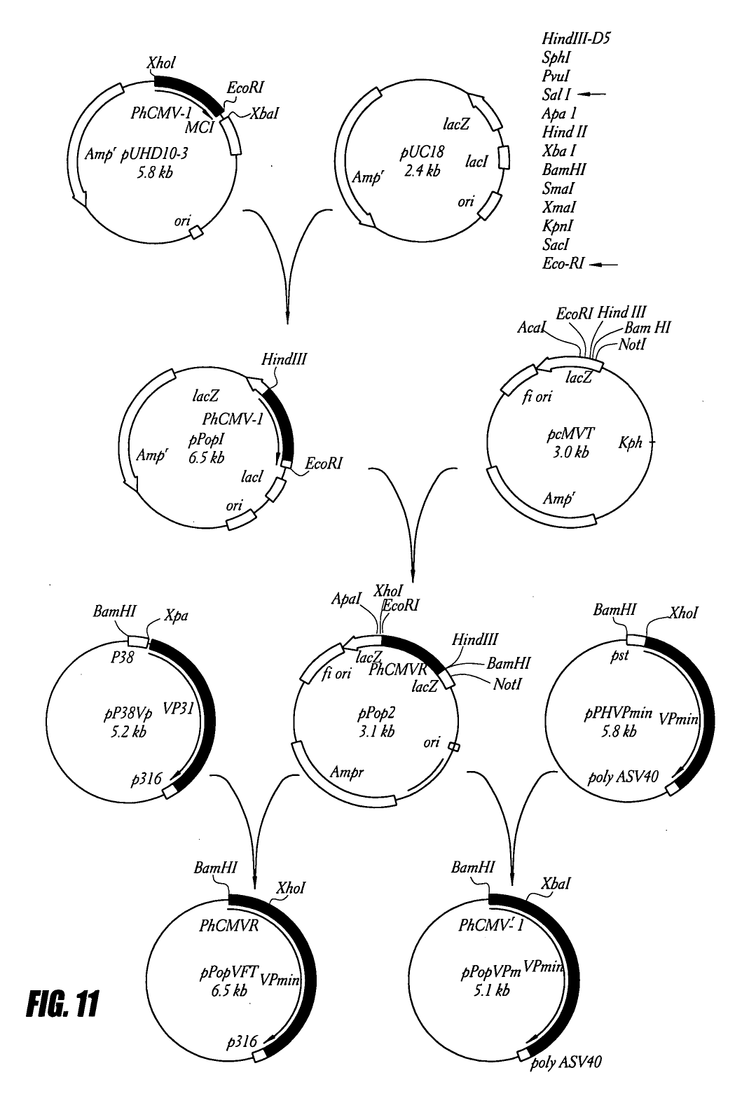

The construction of plasmid ptTA neo is described in FIG. 10.

A XhOI linker is inserted in the SwaI site of PSV2 neo. The resulting plasmid pSV2 neo X is digested by XhoI and Bam HI and the XhoI-Bam HI fragment of pUHD15-1 plasmid containing the tTA transcriptional unit inserted in PSV2 neo resulting in the plasmid ptTA neo.

Selected stably transfected NBK324 clones (G418 selection) were assayed for luciferase expression upon transient transfection (in absence or presence of tetracycline) with the plasmid pUHC13-1 (Gossen and Bujard). The plasmid pUHC13-1 is an expression vector containing the luciferase gene under the control of a promoter activated by tTA. That activation by tTA is completely blocked by tetracycline.

The best clone selected, NBK.MZ.tTA-2, upon electroporation at 230 volts and 1050 μF with 4 μg of pUHC13-1 is charaterized by the following results (expressed in relative luciferase unit rlu/μg protein):

| | |
|---|---|
| 1. mocked electroporation (no plasmid) | 208 |
| 2. electroporation no tetracycline | 793384 |
| 3. electroporation tetracycline 10 ng ml | 19212 |
| 4. electroporation tetracycline 100 ng/ml | 2268 |
| 5. electroporation tetracycline 1 μg/ml | 1568 |

The NBK.MZ.tTA 2 clone was then transfected with the plasmid pPopVPdhfr. That plasmid contains the gene coding for the VP capsids of the MVM under the control of the tTA activated promoter derived from plasmid pUHD10-3 (Gossen and Bujard) and a modified version of the dhfr (dihydrofolase reductase) gene conferring methotrexate (MTX) resistance under the control of the early promoter SV40, derived from the plasmid pSVA3 (Hussain, et al, GENE 112: 179-188, (1992)).

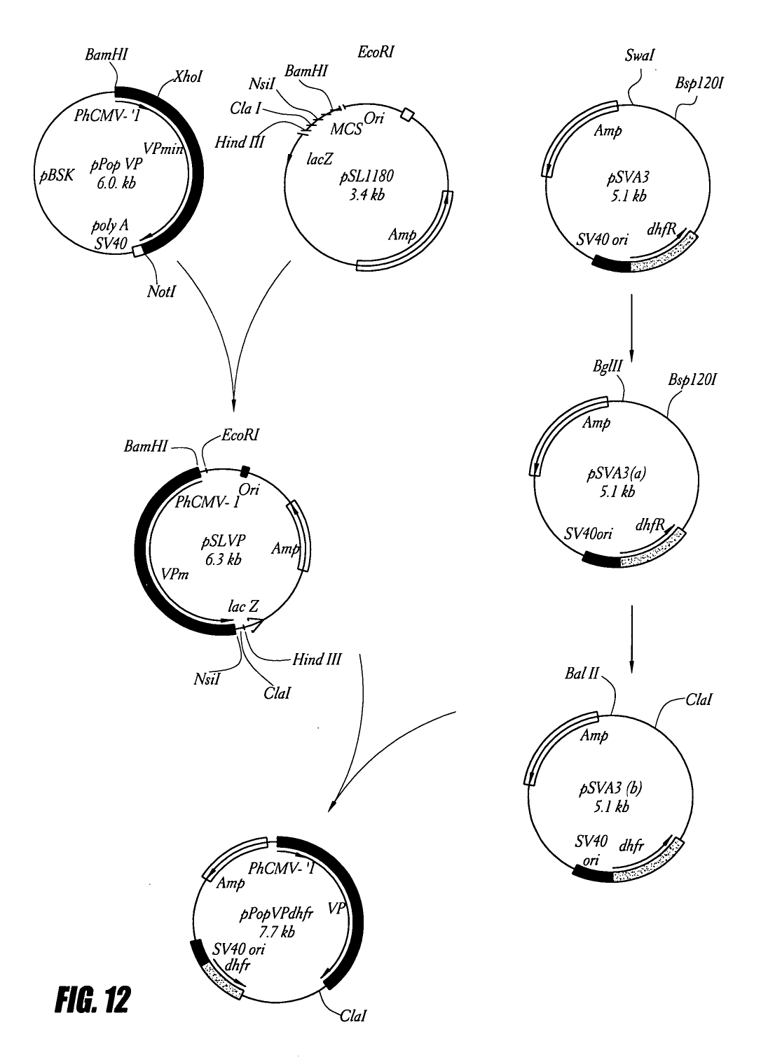

The construction of the plasmid pPopVPdhfr is depicted in FIGS. 11 and 12. Construction of the plasmid P38VPmin

The fragment HindIII-SspI containing the 3′ end region coding for the capsids of MVM in plasmid PMM984 is inserted in the corresponding sites of pUC19. The SspI site is transformed in a NsiI site by the addition of the linker NsiI (5′TGCATGCATGCA-3′ SEQ ID NO: 1). The HindIII-NsiI fragment is inserted in the HindIII et NsiI site of the plasmid pPolyA (where the HindIII site was transformed into a NsiI site) according to the method of Spegelaere P., et al, (Journal of Virology 65: 4919, (1991)) in front of the SV40 polyadenylation signal.

The XbaI-SacI fragment of the resulting plasmid is inserted in the corresponding sites of pBluecript SK(+) (Stratagene, Inc.).

The XhoI-XbaI fragment of PMM984 is inserted in the corresponding sites of the resulting plasmid.

The BamHI-XhoI fragment of plasmid pP38CAT (Caillet-Fauquet et al., EMBO Journal 9: 2989, (1990)) is inserted in the corresponding sites of the resulting plasmid.

The final plasmid pP38Vpmin contains the parvoviral promoter P38, the sequences coding for the VP capsids and the SV40 polyadenylation site.

The fragment XhoI-EcoRI of plasmid pUHD10-3 is inserted into the SalI (compatible with XhoI) and EcoRI sites of pUC18 resulting in the plasmid pPopI.

The fragment EcoRI-HindIII of plasmid pPopI is inserted into the corresponding sites of pGem7 resulting in the plasmid pPop2.

The fragment BamHI-XhoI of plasmid pPop2 is inserted into the corresponding sites of pP38Vpmin resulting in the plasmid pPopVPmin. Construction of the plasmid pSVA3(b):
by the addition of a Bgl II site linker at the SwaI site resulting in the plasmid pSVA3(a)
a ClaI site is introduced into the plasmid pSVA3(a) by addition of a ClaI linker at the Bsp1201 site resulting in the plasmid pSVA3(b). Construction of the plasmid pSLVP

The fragment BamHiI-NsI of the plasmid pPopVPm was inserted in the corresponding site of the plasmid pSL1180 (Broscius, Journal DNA 8: 759, 1989) resulting in the plasmid pSLVP. Construction of the plasmid pPopVPdhfr

The fragment BamHI-ClaI of the plasmid pSLVP was inserted into the sites Bgl II (compatible with BamHI) and ClaI of plasmid pSVA3(b)) resulting into plasmid pPopVPmdhfr.

Upon stable transfection with the pPopVPdhfr in the presence of G418, tetracycline and increasing amounts of MTX of the NBK.MZ.tTA2 cell line, 2 clones survived in the selection procedure. The NBK.MZ.tTA.VP-1 and NBK.MZ.tTA.VP-2 cell lines were maintained in culture in the presence of G418, MTX and tetracycline. Upon transfection with MVM-B7-1 plasmid in absence of tetracycline, they produce recombinant virions at titres comparable with those obtained by the co-transfection method.

However, it is noteworthy that no wild type contamination could be detected by in situ hybridization.

Upon concentration, according to the method of Avalosse, B. et al (Journal of Virological Methods 62: 179-183, (1996)) high titres (5.10⁸ IU/ml) could be obtained according to the methods of the present invention.

These concentrations are high enough for the preparation of a pharmaceutical composition used for in-vivo treatment upon animals (including humans).

### EXAMPLE 13

### Construction of a Recombinant Parvovirus Used for the Transduction of Non-transformed Cells

The construct consists of a parvovirus having the Hind III (2650)-XbaI(4342) sequence replaced by a sequence coding for an effector gene and having a promoter sequence inserted at a site NCoI(259), the promoter sequence being a sequence derived from a promoter active in the target cell. The advantages of such a construct is that in a non-transformed cell, the parvoviral promoter is silent, so an active promoter is inserted upstream from the coding sequence of the NSI proteins. So even in non-transformed cells, the NSI protein, which is necessary for transactivation of the P38 promoter and for the amplification of the viral DNA, will be synthesized and that will lead to a very abundant synthesis of the effector gene.

In the present example, the following construct has been made. The CMV-IE promoter was inserted at the NCoI site and the green fluorescent protein gene (GFP) was used as a reporter gene. This plasmid is named pMZ.MVM.CMV.GFP.

The insertion of the GFP genes into the parvoviral vector comprises the steps of:
The 789 bp fragment Hind III-XbaI of plasmid pEGFP-NI® (Clontech) is inserted into the sites Hind III (2650) and XbaI (4342) of PMM984 resulting in the plasmid pMZ.MVM.GFP.
The 591 bp EcoRV fragment of PMM984 is inserted into the EcoRV site of plasmid LITMUS 38® (New England Biolabs) resulting in the plasmid Litmus-EcoRV-MVM).

The CMV promoter (38-682) including the Kozak consensus translation initiation site was amplified by PCR with the PWO Polymerase® (Boehringer) with the following primer:

| | |
|---|---|
| 5′ CCATGGCATAGCCCATATATGGAGTTCCGCG 3′ | (SEQ ID NO:2) |
| 5′ TTGCTCACCATGGTGGCGA | (SEQ ID NO:3) |

The blunt PCR product is inserted into the EcoRV site of the plasmid LITMUS and the resulting plasmid is amplified and digested by NCoI. The 649 bp NCoI fragment is then inserted into NCoI site of the plasmid LITMUS-EcoRV-MVM resulting into the plasmid Litmus-MVM-CMV.

The EcoRV fragment of Litmus-MVM-CMV (1240 bp) is inserted into the 2 EcoRV sites of the plasmid pMZ.MV.GFP resulting in the plasmid pMZ.MVM.CMV.GFP.

Upon transfection of normal cells with either pEGFP-NI (Expression Vector containing the GFP gene under the control of the MV promoter) or with pMZ.MVM.CMV.GFP, pMz.MVM.CMV.GFP. was always superior to pEGFP-NI both in terms of number of transduced cells (fluorescent) and in term of level of transduction (level of fluorescence) as measured with a fluorescence cell sorter.

Upon transfection of packaging cell line with pMZ.MVM.CMV.GFP or upon co-transfection of NBK324 or COS cells with a helper plasmid providing the VP capsids genes in trans, recombinant virions MVM.CMV.GFP were produced.

These virions were able to transduce very efficiently normal cells and transformed cells.

It is also possible to use other effector genes as described in the previous constrictions.

What is claimed is:

1. A first nucleotide sequence comprising a second nucleotide sequence and a third nucleotide sequence, said second nucleotide sequence comprising the nucleotide sequence of an oncoselective autonomous parvovirus, and said third nucleotide sequence comprising at least one effector nucleotide sequence encoding an effector polypeptide, wherein said effector polypeptide effects the destruction or the normalization of cancer cells,
wherein the effector nucleotide sequence comprises at least one sequence chosen from the group consisting of:
a nucleotide sequence that encodes a cytotoxic polypeptide or at least one fragment of this polypeptide, and
a nucleotide sequence that encodes a molecule which confers on a transfected cell sensitivity to a radioactive toxic agent.

2. The first nucleotide sequence according to claim 1, wherein the second nucleotide sequence comprises the nucleotide sequence of a oncoselective autonomous parvovirus which is chosen from the group consisting of parvovirus H1 and a fibotropic parvovirus variant of Minute virus of Mice (MVMp).

3. The first nucleotide sequence according to claim 1, wherein the second nucleotide sequence comprises a nucleotide sequence of an oncoselective autonomous parvovirus which lacks nucleotide sequences encoding the parvovirus capsid proteins VP1 and VP2.

4. The nucleotide sequence according to claim 3, further comprising inserted between a promoter P4 and a non-structural protein NSI, a promoter which is activated in target cells.

5. The nucleotide sequence according to claim 3, wherein the nucleotide sequence of the oncoselective autonomous parvovirus further lacks a nucleotide sequence of a promoter P38 and nucleotide sequences encoding the parvovirus nonstructural proteins NSI and NSII.

6. The first nucleotide sequence according to claim 1, wherein said third nucleotide sequence comprises an effector nucleotide sequence which comprises at least two coding nucleotide sequences, at least two non-coding nucleotide sequences, or combinations thereof, operably linked in polycistronic subunits under the control of a single promoter unit.

7. The nucleotide sequence according to claim 6, wherein the effector nucleotide sequence is between two coding nucleotide sequences and the effector nucleotide sequence comprises one IRES nucleotide sequence.

8. The first nucleotide sequence according to claim 1, wherein the third nucleotide sequence comprises an effector nucleotide sequence which encodes at least one fusion polypeptide containing at least one ligand selected from the group consisting of a hypervariable end of an antibody, a cytokine and a growth factor, wherein the ligand binds specifically to at least one molecule expressed at the surface of cancerous or infected cells.

9. The first nucleotide sequence according to claim 1, wherein the fragment of the cytotoxic polypeptide encoded by said effector nucleotide sequence is fragment A of diphtheria toxin.

10. The first nucleotide sequence according to claim 1, wherein the molecule encoded by the effector nucleotide sequence is Herpes simplex virus type 1 thymidine kinase (HSV-TK), and the radioactive toxic agent is a guanosine analog labeled with a radioisotope which emits Auger electrons.

11. The first nucleotide sequence according to claim 1, wherein the effector nucleotide sequence comprises at least one nucleotide sequence which can be transcribed into an RNA, which destroys or normalizes cancer cells or infected cells.

12. The nucleotide sequence according to claim 11, wherein the nucleotide sequence which can be transcribed into an RNA which destroys or normalizes cancer cells or infected cells is an antisense RNA or a ribozyme.

13. The first nucleotide sequence according to claim 1 which further comprises at least one regulatory nucleotide sequence activated by transactivation factors specific for a medical condition and/or for the affected cellular tissue and which cisactivates the effector nucleotide sequence.

14. The nucleotide sequence according to claim 13, wherein the regulatory nucleotide sequence contains at least one promoter and/or at least one enhancer transactivatable in certain specific tissues and chosen from the group consisting of:
a nucleotide sequence controlling the expression of the gene encoding α-fetoprotein (AFP),
a nucleotide sequence controlling the expression of human placental protein 11 (PP11),
a nucleotide sequence controlling the expression of antigen CO-029,
a nucleotide sequence controlling the expression of antigen H23,
a nucleotide sequence controlling the prostatic expression of prostatic secretory protein PSP94,
a nucleotide sequence controlling the expression of the protein pHGR11 associated with melanoma, ovarian cancer, adenocarcinoma of the colon and of the prostate,
a nucleotide sequence controlling the expression of protein pHGR74, expressed in the testicles, the prostate, the seminal vesicle and the granulosa of the ovary,
nucleotide sequences controlling the expression of proteins specific for the mammalian epithelium,
a nucleotide sequence controlling the expression of tyrosinase, expressed in the melanocytes and malignant melanoma,
nucleotide sequences controlling the expression of elastase, expressed only in the exocrine pancreas, and
a nucleotide sequence controlling the hypophysial expression of prolactin.

15. A recombinant vector comprising the sequence or a portion of the first nucleotide sequence according to claim 1.

16. A nucleotide sequence comprising the nucleotide sequence of an autonomous parvovirus, and at least one effector nucleotide sequence encoding a polypeptide which effects the destruction or normalization of cells infected by intracellular infectious parasites,
wherein the effector nucleotide sequence comprises at least one sequence selected from the group consisting of:
a nucleotide sequence that encodes a cytotoxic polypeptide or at least one fragment of this polypeptide, and
a nucleotide sequence that encodes a molecule which confers on a transfected cell sensitivity to a radioactive toxic agent.

17. The nucleotide sequence of claim 16, wherein said mammalian epithelium is uterine epithelium.

18. The nucleotide sequence of claim 10, wherein said radioisotope is ¹²³Iodine.

19. The nucleotide sequence of claim 1, wherein the effector nucleotide sequence comprises at least one nucleotide sequence which is transcribed into an RNA being a ribozyme, said nucleotide sequence being placed at the ASE I restriction site in the small intron of the MVMp genome.

20. The nucleotide sequence of claim 1, wherein the effector nucleotide sequence comprises at least one nucleotide sequence which can be transcribed into a RNA being a ribozyme and which comprises the addition in 3′ of a sequence that protects athe ribozyme from digestion by 3′ exonucleases.

21. The nucleotide sequence of claim 20, wherein the effector nucleotide sequence comprises the addition in 3′ of a transcription terminator sequence of the T7 bacteriophage.


SEQUENCE LISTING (3 sequences)

SEQ ID NO: 1 - DNA, 12 bp, Artificial Sequence
  Nsi1 linker
tgcatgcatgca

SEQ ID NO: 2 - DNA, 31 bp, Artificial Sequence
  Primer
ccatggcatagcccatatatggagttccgcg

SEQ ID NO: 3 - DNA, 19 bp, Artificial Sequence
  Primer
ttgctcaccatggtggcga